(19) United States
(12) Patent Application Publication
(10) Pub. No.: US 2011/0137331 A1
(43) Pub. Date: Jun. 9, 2011

(54) Perfusion device

(76) Inventors: Michael Walsh, County Limerick (IE); Barry O'Connell, Limerick (IE); Timothy McGloughlin, County Limerick (IE); Michael V. Lawlor, Limerick (IE); Michael O'Donnell, Country Tipperary (IE)

(21) Appl. No.: 12/926,704

(22) Filed: Dec. 6, 2010

Related U.S. Application Data
(60) Provisional application No. 61/282,034, filed on Dec. 7, 2009.

(51) Int. Cl. A61M 29/02

(52) U.S. Cl. 606/194

(57) ABSTRACT

A vascular filter device (1) has a perfusion balloon (3) with a through-hole for blood flow. There is a distal filter (4) for trapping emboli, and the filter (4) is configured to filter an annular cross-sectional area extending from a vessel wall, while leaving an un-filtered central passageway. The filter (4) may be substantially frusto-conical in shape when deployed, and have a proximal tubular part (41) which is attached to the perfusion balloon or a support for the perfusion balloon. The filter has a retainer (5) to retain an end of the filter in an in-use position abutting a vessel wall. The retainer may comprise wires (5) or an inflated ring (8). The device may have an internal support (2, 12, 22, 96, 112) for the perfusion balloon (3, 11, 21, 95, 111). The balloon inflates and expands the inner arterial wall, and the filter captures emboli released during the procedure while leaving a channel that allows blood to flow to tissues distal to the blockage, maintaining antegrade flow. This may prevent the onset of an ischemic stroke or other perioperative neurological events.

### INTRODUCTION

1. Field of the Invention

The invention relates to medical devices, and more particularly to devices for insertion in blood vessels and having a perfusion balloon for expanding to press against the vessel wall.

2. Prior Art Discussion

The balloon catheter is well established as an important medical device. The device can be used on its own to open a blocked artery or it can be used to deliver a stent (a small wire mesh tube) to the offending artery. The stent will expand when the balloon expands and is left in the artery acting as a scaffold to keep it open.

Carotid angioplasty and stenting is being used widely to treat severe carotid obstructive disease. An acute complication of carotid angioplasty and stenting relates to the distal embolisation of plaque particles during the endovascular procedure, with the risk of definitive stroke. Studies using in vivo monitoring (such as Magnetic Resonance Imaging (MRI), Computed Tomography (CT) or Transcranial Doppler) have shown that carotid stenting is associated with a higher incidence of transient ischaemic attack (TIA) or stroke from embolisation of plaque fragments in comparison to surgical endarterectomy. To reduce the possibility of these plaque fragments causing periprocedural neurological complications, cerebral protection is used. Embolic protection has also been used in other arteries, such as coronary and renal arteries, and therefore the technology is not limited to arteries belonging to the carotid bifurcation.

EP1400257 (Embol-X Inc) describes a percutaneous stent deployment assembly comprising a balloon and a filter for capturing emboli. It includes a conical-shaped filter which extends across the full cross-sectional area of the vessel. U.S. Pat. No. 5,545,135 describes a perfusion balloon stent comprising a balloon which is annular in cross-section enabling blood flow through its central passageway.

The invention is directed towards providing an improved perfusion device for applications such as treatment of blocked blood vessels, with reduced disruption of blood flow during the surgical procedure and/or with limiting of the host tissue response which would reduce flow due to perceived high blood pressure.

### SUMMARY OF THE INVENTION

According to the invention, there is provided a vascular device comprising:
a perfusion balloon having, when inflated, a through-hole for blood flow, and
a distal filter for trapping emboli,
wherein the filter comprises a filtering portion which is configured to filter an annular cross-sectional area extending from a vessel wall while leaving an un-filtered central passageway.

In one embodiment, the filtering portion is configured to, when deployed, extend distally from the perfusion balloon and then in a direction which is at least partly radial, and then in a direction which is at least partly proximal.

In one embodiment, the filtering portion is substantially frusto-conical in shape when deployed.

In one embodiment, the filter has a proximal stem which is attached to the perfusion balloon or a support for the perfusion balloon.

In one embodiment, the filtering portion has an asymmetrical edge for improved retrieval to enable improved device deliverability and extraction.

In one embodiment, the filter comprises a retainer adapted to retain an end of the filtering portion in an in-use position abutting a vessel wall.

In one embodiment, the retainer comprises members extending from the perfusion balloon or a support for the perfusion balloon.

In one embodiment, the members include wires.

In one embodiment, the retainer is adapted to apply a radially outward bias to urge the filter to abut a vessel wall.

In one embodiment, the retainer comprises cantilevered members.

In one embodiment, the retainer is ring-shaped having a configuration conforming to a vessel perimeter.

In one embodiment, the retainer is inflatable.

In one embodiment, the retainer is linked with the balloon for simultaneous inflation.

In one embodiment, the device further comprises an internal support for the perfusion balloon.

In one embodiment, the support is adapted to allow gradual and controlled inflation of the perfusion balloon.

In one embodiment, the support comprises a scaffold structure.

In one embodiment, the scaffold structure comprises elements of a shape memory material.

In one embodiment, the support comprises at least one element in the form of opposed arches.

In one embodiment, the support comprises an element in the form of a coil.

In another embodiment, the support comprises an inner balloon adapted to fit within the perfusion balloon.

In one embodiment, the inner and perfusion balloons are coiled, the coil pitches being in or out of phase.

In one embodiment, the device is adapted to support a stent around the perfusion balloon and a mechanism for delivery of the stent by inflation of the perfusion balloon.

In another aspect, the invention provides a method of performing a procedure using a vascular device comprising a perfusion balloon having, when inflated, a through-hole for blood flow, and a distal filter for trapping emboli, the filter being configured to filter an annular cross-sectional area extending from a vessel wall while leaving an un-filtered central passageway, the method comprising the steps of:
inserting the device into a vessel,
inflating the perfusion balloon while allowing blood to flow through the through-hole,
the filter capturing emboli which are dislodged from the vessel wall while allowing un-impeded blood flow through the central passageway

In one embodiment, the filter is supported by retainers to engage the vessel wall.

In one embodiment, the retainer is inflatable and is inflated together with the perfusion balloon so that it engages around the vessel wall.

### DETAILED DESCRIPTION OF THE INVENTION

The invention will be more clearly understood from the following description of some embodiments thereof, given by way of example only with reference to the accompanying drawings in which:—

FIG. 1 is a cut-away perspective view of a perfusion device of the invention when deployed, the device having an annular balloon and a support rib cage balloon and an embolic protection filter;

FIG. 2 is a partly cut-away perspective view showing an alternative filter, having a wall apposition support mechanism using an inflated ring balloon;

FIG. 3 is a cut-away perspective view of an alternative device, in this case having a metallic support coil;

FIG. 4 is a partly cut away view showing part of an alternative device, in this case having a mechanical support with arched metal members when fully deployed;

FIG. 5 is a longitudinal cross-sectional view showing site of occlusion of plaque B within an artery wall A;

FIG. 6 is a longitudinal cross-sectional diagram showing positioning of the device of FIG. 3; and

FIG. 7 is a similar view showing deployment of the device in which a catheter sheath is retracted thus deploying the filter support wires and the filter mesh;

FIGS. 8 and 9 show inflation of the device in which the support inhibits collapse of the inner surface of the perfusion balloon and supports the outward inflation of the balloon and hence deformation of the occlusion and release of emboli which are captured by the filter; and

FIG. 10 shows retrieval of the device;

FIG. 11 is a diagrammatic cross-sectional view showing the device of FIG. 1 when deployed, in which operation of the rib cage balloon is illustrated;

FIG. 12 is a set of three views showing a filter for a device of various embodiments and a cross-sectional view of an alternative filter, these views illustrating that the filter screens emboli only along an annular cross section with remaining blood being allowed to perfuse uninhibited through the lumen of the filter;

FIG. 13 is a plot showing competing physical phenomena due to the presence of a support scaffold, in which as the inner lumen diameter increases to relieve the fluid flow pressure drop across the device, the associated strain applied to the occluding plaque also increases due to increasing diameter;

FIG. 14 is a plot showing stress and strain uniaxial characterisation tests of fresh human carotid plaque with varying plaque type and stiffness;

FIG. 15 is a plot showing strain at the inner and outer diameters of an internal carotid artery (ICA) associated with dilation of a 1 and 2 step dilation balloon;

FIG. 16 is a plot showing velocity profiles along the centre of a vessel for three device configurations, in which a filter region is highlighted within a box and the direction of blood flow is noted with an arrow, and FIG. 17 shows velocity contours and direction for the three device configurations of FIG. 16;

FIG. 18 is a cut-away cross-sectional view showing a part of another embodiment of filter for devices of the invention, the filter having an alternative filter wall apposition support mechanism;

FIG. 19 is a cut-away cross-sectional view showing a part of a device of another embodiment, having an inner rib cage support balloon and an outer coiled balloon;

FIG. 20 is a cut-away cross-sectional view showing a part of a device of another embodiment, having an inner coiled support balloon and an outer coiled perfusion balloon;

FIG. 21 is a cut-away cross-sectional view showing a device of another embodiment of the invention, demonstrating that an inner coiled support balloon and an outer coiled perfusion balloon can have different pitch angles and orientation in relation to one another; and

FIGS. 22 and 23 are cut-away cross-sectional views showing parts of alternative devices having inner and outer coiled support and perfusion balloons.

### DESCRIPTION OF THE EMBODIMENTS

### Overview

Devices of the invention in various embodiments include an annular embolic filter for embolic protection device combined with a perfusion balloon. When the balloon is deployed, the filter allows for emboli-free blood to flow through the region of treatment. Blood is allowed to pass uninhibited during the treatment, while at the same time the filter protects the cranial vascular system from emboli dislodged from the artery wall upon inflation of the perfusion balloon. This results in a minimum loss in blood pressure during treatment, in contrast to many prior devices having filters which extend across the full cross section. Also, the latter devices may result in losses in blood pressure to vessels distal of the treatment site as the filter captures and becomes saturated with emboli. Thus, by providing, in a counter intuitive manner, a filter with a hole in the middle of the device, optimum emboli filtration can be achieved and loss of blood pressure can be minimised during deployment.

### DESCRIPTION WITH REFERENCE TO THE DRAWINGS

Referring to FIG. 1 a perfusion device 1 of the invention comprises an inner support structure 2, an outer inflatable balloon 3, and an embolic protection filter 4. The support structure 2 is a balloon having a rib-cage configuration, and the outer balloon 3 has an elongated annular configuration. The filter 4 has a membrane which extends along the length of both balloons, providing stability for the balloons and also offering further protection during the balloon/stent deployment of the device. Importantly, the filter 4 covers only an annular cross-sectional region, having a central passageway to allow emboli-free blood to flow and being folded back on itself adjacent the vessel wall creating a basket type effect and held in place by stays 5. The latter are of a metal which is biased radially outwardly to retain the filter mesh open. The annular filter 4 extends from the distal end of the device, and is retained in its annular shape by the support wires 5, which are of a shape memory alloy, extending from the distal end of the balloon.

FIG. 2 shows an alternative filter support 8, namely a ring balloon structure inflated to maintain artery wall contact during the procedure. This ‘filter ring’ can have standalone inflation or inflation can be initiated by inflation of the inner balloon 2 and will provide full lumen wall apposition.

FIG. 3 illustrates a device 10 of an alternative embodiment. There is an elongate outer annular balloon 11 and an inner support structure 12. The inner support structure 12 consists of a shape memory support coil, which is not attached to the outer balloon 11. The inner support coil 12 acts as support to the outer annular balloon 11 to prevent collapse of the inner diameter of the outer balloon 11.

FIG. 4 illustrates a perspective cut-away view of a device 20 of an alternative embodiment. There is an elongate outer annular balloon 21, and a mechanical inner support 22 on a catheter shaft 23. The inner support structure 22 consists of a radial array of support arches, made from a shape memory alloy that can be distended when required to support the outer annular balloon 21, and retracted when not in use.

FIGS. 5 to 10 are longitudinal and transverse cross-sectional views showing deployment and removal of the device of FIG. 3 in an occluded artery.

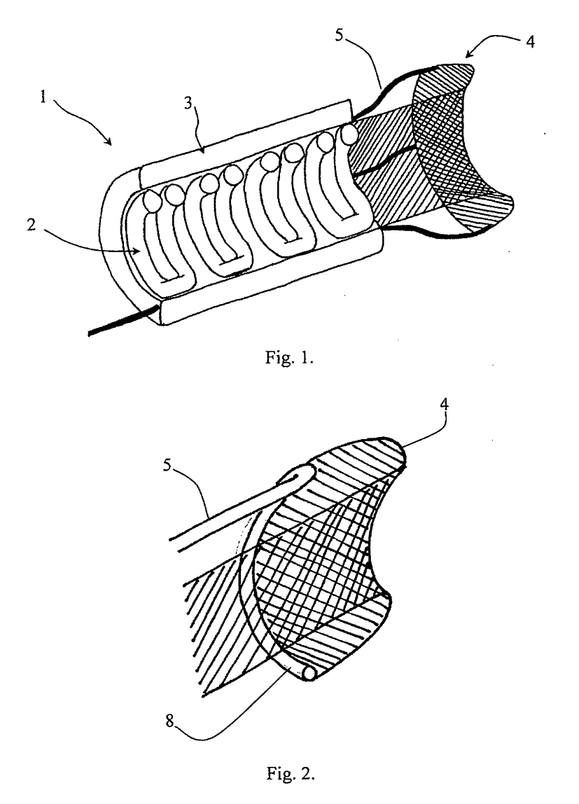

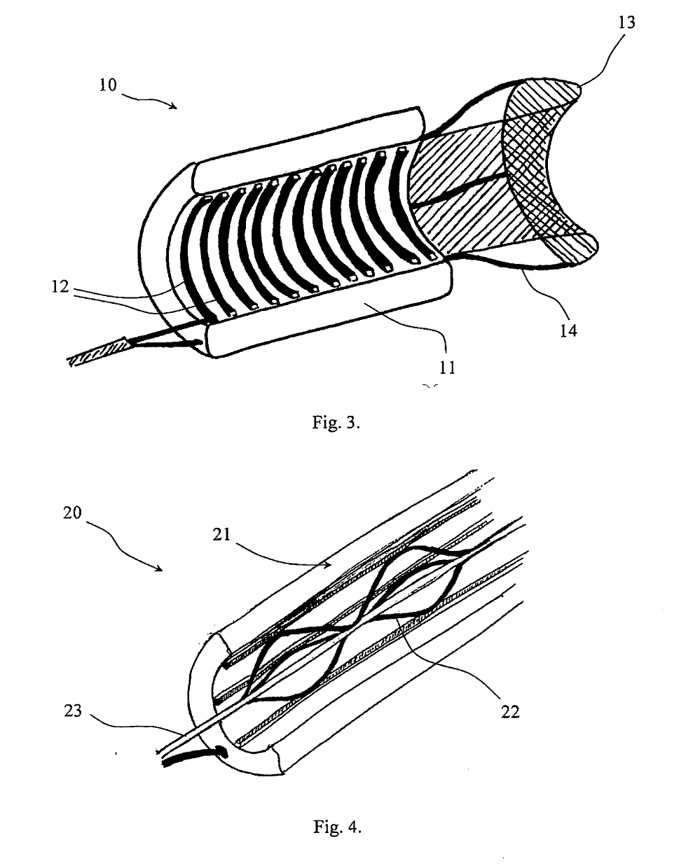

### Stage 1

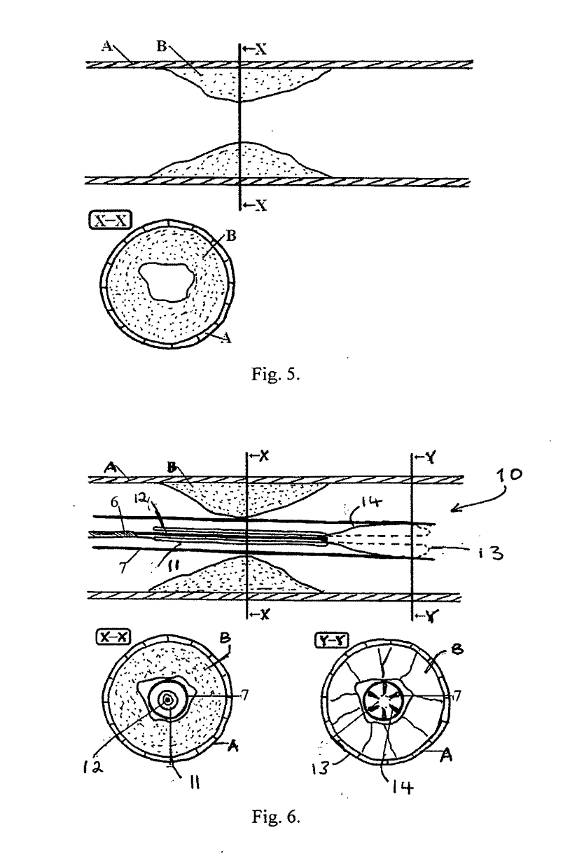

FIG. 5 shows an occluded arterial vessel A. Prior to deployment of the device 10, a guide-wire must navigate through the arterial network and traverse this occlusion to facilitate the safe deployment of the balloon and filter.

### Stage 2

Once the guide wire is in place, the device 10 with the perfusion balloon 11 is positioned within the site of the occlusion B and the filter 13 distal of the occlusion site, FIG. 6.

### Stage 3

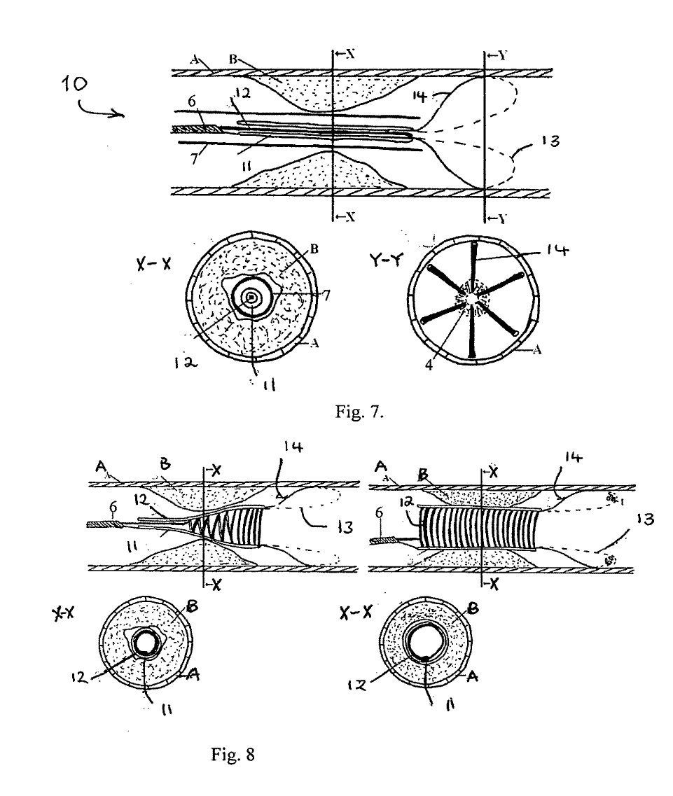

Once the device is in place, the catheter sheath 7 is retracted. As the catheter sheath 7 is removed, the filter 13 is deployed, FIG. 7. It is envisaged that the filter support wires 14 are of a shape memory alloy, so that when the catheter sheath 7 is removed, the filter wires 14 deform to their predefined shape and orientation. An alternative mechanism of filter wall contact can also be used. The number of filter support wires can vary and can be equally spaced in the circumferential direction, FIG. 7—cross-section Y-Y. The maximum number of support wires 14 is limited to the trackability and stiffness of the device within the catheter. This encapsulates the site of the occlusion and thus captures any emboli released during the angioplasty procedure.

### Stage 4

Once the catheter sheath 7 is removed, the inner support can be deployed, FIG. 8. The support coil 12 of the device 10 is of a shape memory alloy and prior to deployment, the coil 12 is in a stretched configuration within the catheter. As the coil is unwrapped the coil takes to its pre-defined shape. The coil 12 acts as an initial angioplasty device, deforming the offending plaque/disease/occlusion B prior to inflation of the outer balloon 11. As the plaque/disease/occlusion B deforms at this stage, the perfusion filter device is in place to capture released emboli. The deployment of the support coil 12 immediately permits the flow of blood beyond the catheter and retains it for the entire procedure. If an alternative balloon support of the device, as per FIGS. 1, 19, 20, and 21, were to be used it would also be deployed at this stage, prior to the inflation of the outer balloon.

### Stage 5

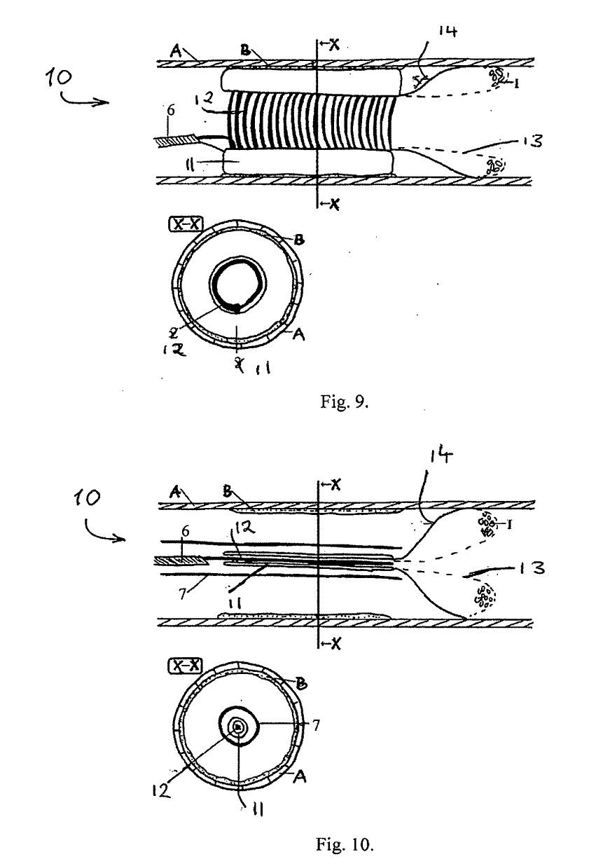

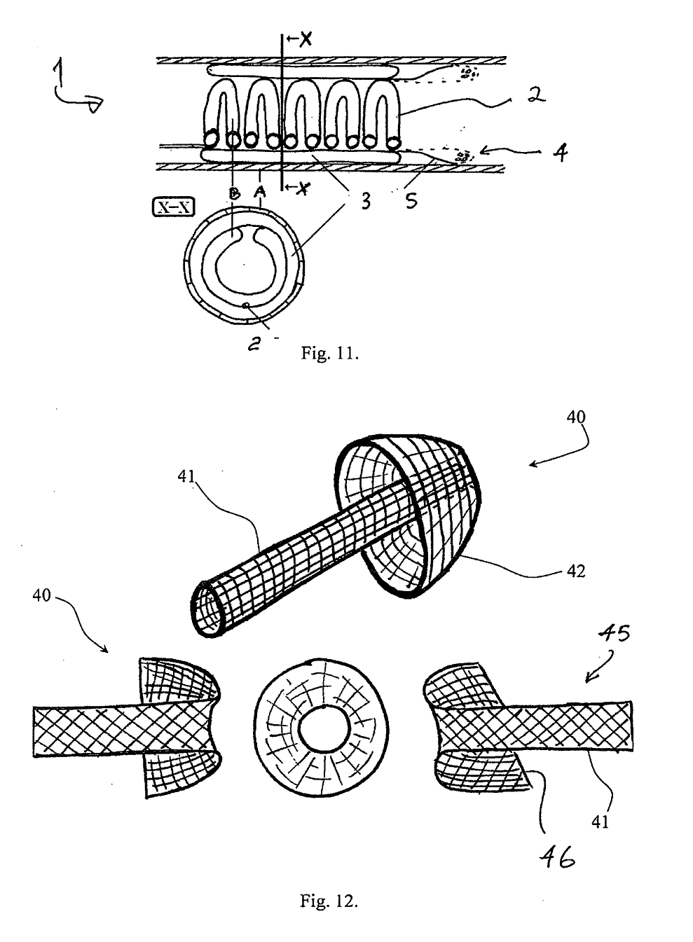

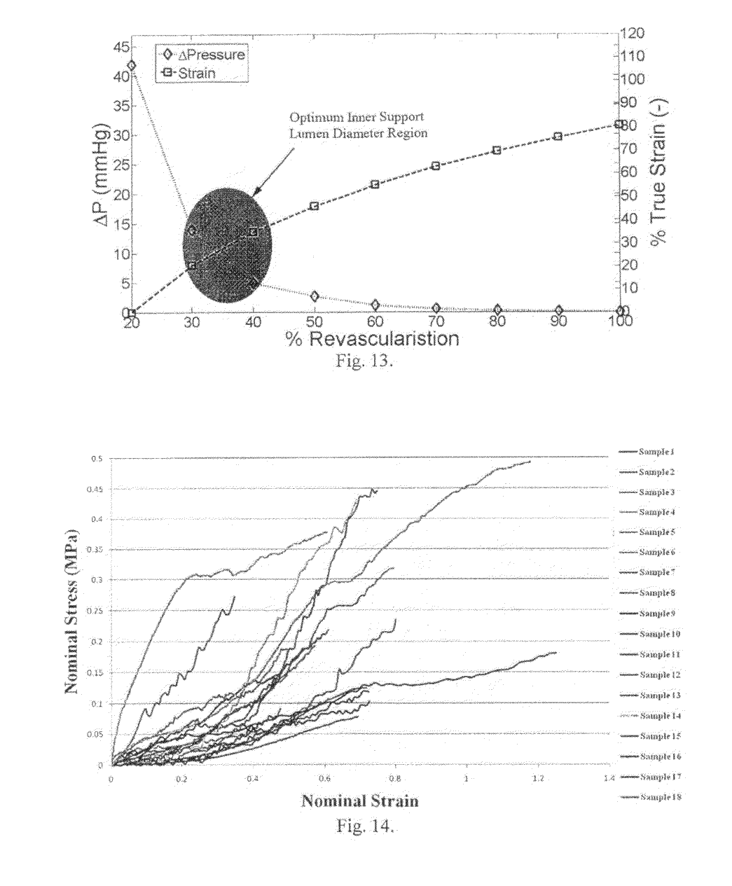

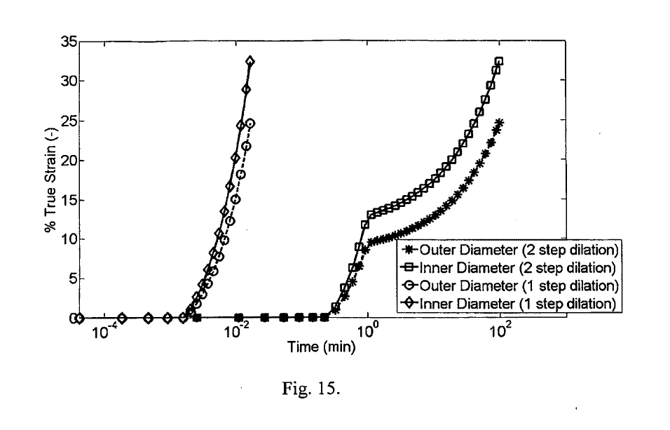

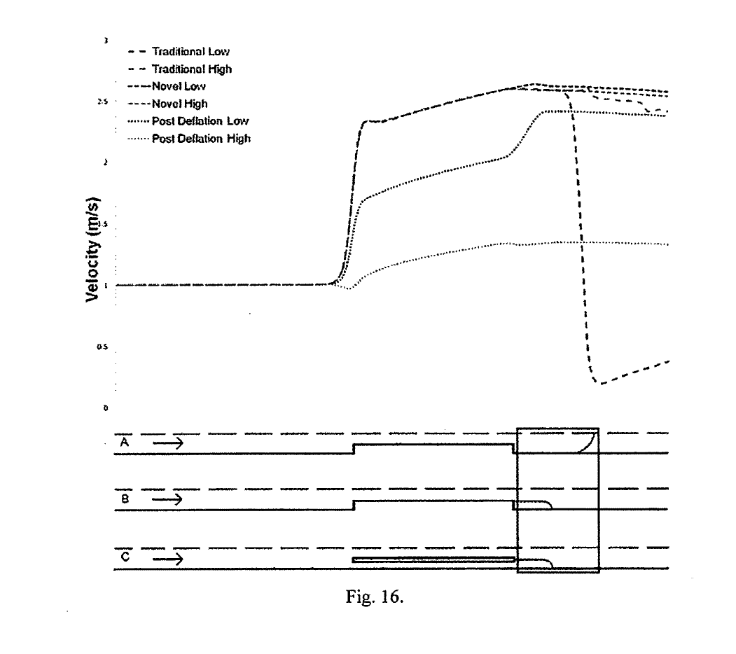

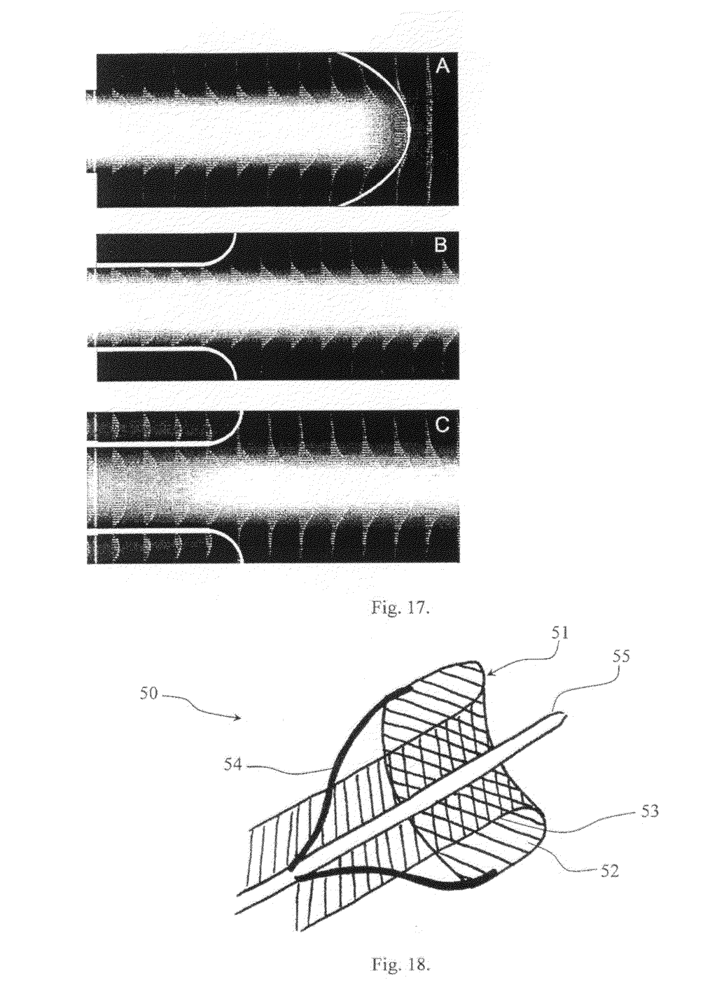

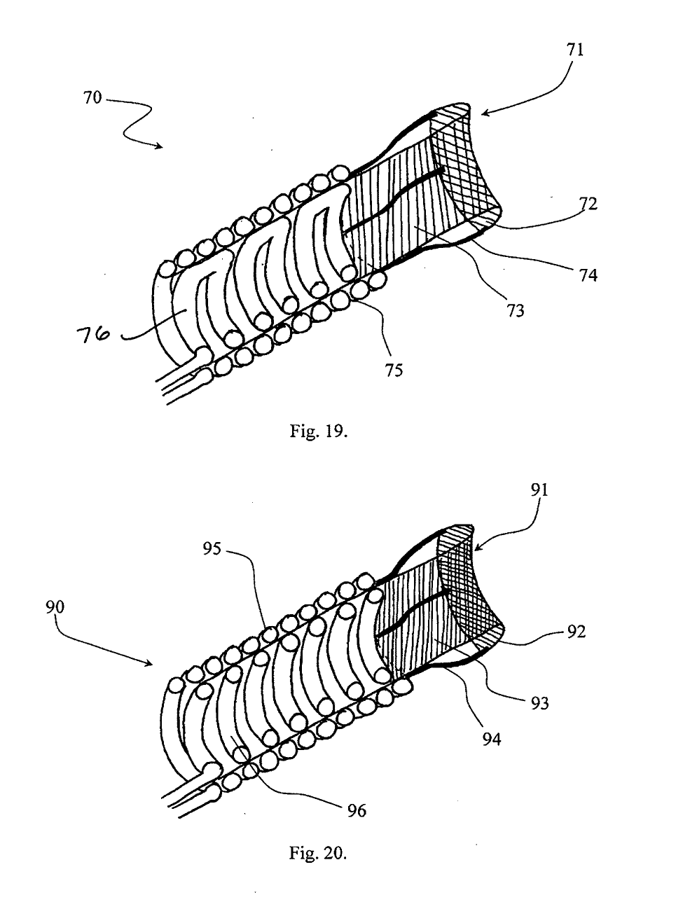

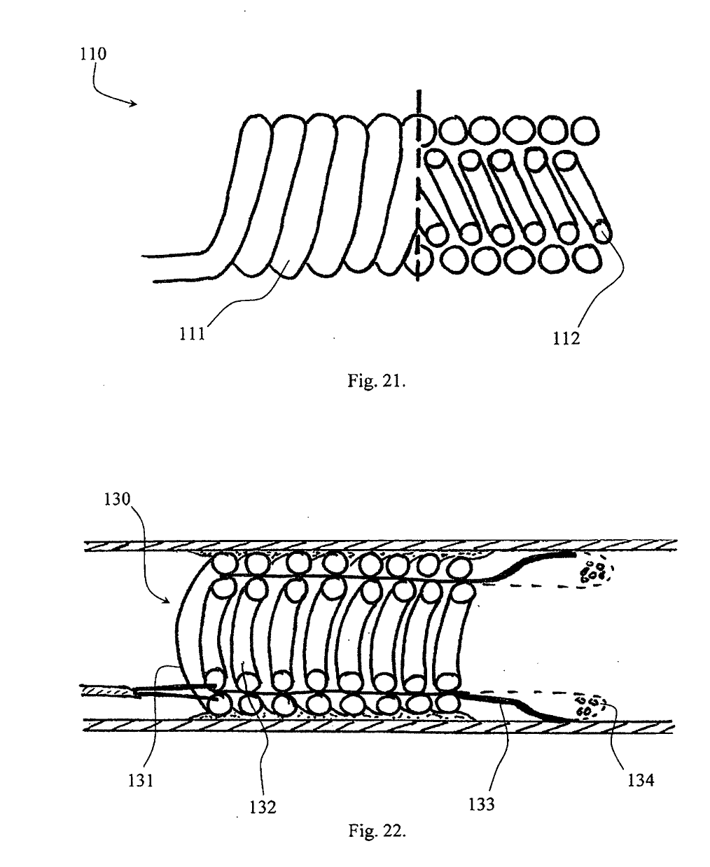

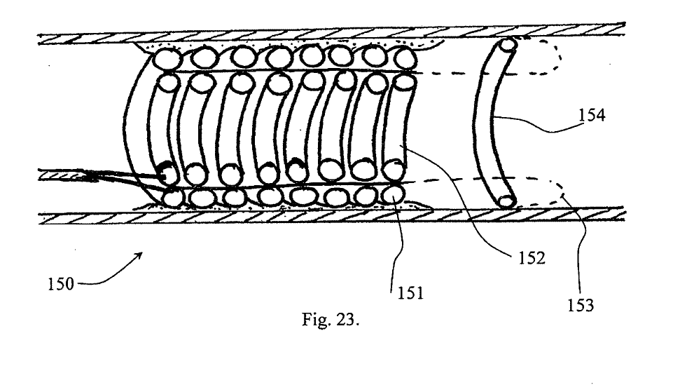

Once the support coil 12 is deployed the balloon 11 can be inflated, FIG. 9. Gradual inflation of the outer balloon 11 is possible due to the support coil, or other support mechanism, retaining a lumen for continuous antegrade blood supply. The process of controlled inflation is important as current angioplasty techniques using standard balloons result in more frequent persistent hypotension in patients. This is due to the fact that during current methodologies of these procedures, the balloon angioplasty devices over-stretch the arterial wall/plaque/diseased artery, inducing para-sympathetic signals from the host tissue, reducing the work rate of the heart and increasing hypotension of the patient. Overstretching of the vessels is a default of current carotid angioplasty procedures due to the small window of time for cranial blood flow to be stopped. Gradual inflation of the perfusion balloon allows time for the host tissue to adjust to these pressures and stretches, and also reduces the potential for recoil of the arterial vessel once the perfusion balloon is removed.

### Stage 6

After a desired inflation period the device 10 can be removed. The perfusion balloon 11 is deflated and the support coil 12 is retracted. The catheter retrieval sheath 7 is deployed to encapsulate the perfusion balloon 11. The device remains deployed for a period of time after the collapse of the perfusion balloon 11 as to capture any emboli dislodged during the angioplasty procedure. The filter 13 containing any plaque is then collapsed by further deploying the catheter retrieval sheath 7. Once the catheter retrieval sheath 7 fully encapsulates the embolic protection system, the entire device is removed, leaving a patent artery in its wake.

FIG. 11 illustrates the full deployment of the device 1 of FIG. 1 with the rib cage balloon support 2.

FIG. 12 illustrates an annular embolic protection filters 40 and 45 for use in various embodiments. In the geometrical design of the filter 40 the outer diameter is normal to the lumen surface, and in the filter 45 the outer diameter of the filter basket is asymmetrical, at an angle to the surface of the artery wall, creating an elliptical edge, which may improve device functionality in a number of areas. The filter 45 creates an elliptical contact with the artery wall due to the development/formation of a contact slope/angle on the filter basket. This basket angle may be used to aid in device crimping prior to deployment, to improve stability and wall apposition within the artery due to an increase in artery wall contact, and to improve deliverability and ease of withdrawal into the catheter sheath. This filter basket angulation is achieved in the manufacturing/production of the device and is not as a result of its deployment.

The preferred filter materials are including but limited to; compliant polymers such as polyurethane, Latex, Silicone, polyethylene, polyethylene terephthalate (PET), nylon, or shape memory materials such as superelastic nickel titanium alloy, or stainless steel. The filter material may also include an anti-thrombogenic coating. A second material is also envisaged containing a metallic element which can be visualised within the blood vessel using fluoroscopic imaging or angiography imaging, which will act as a locator to the operator.

FIGS. 13 to 17 demonstrate the performance of various aspects of the device and also the behaviour of the arterial plaques within the arteries.

The design criterion for the support scaffold is a function of initial strain applied to the occluding plaque, flow rate and pressure drop across the length of the device. As a result, the support scaffold is designed to minimise the impacts of these physical phenomena on the performance of this device. A large inner support lumen diameter is required to minimise the pressure drop (ΔP) across the device. However, as the lumen diameter increases, the result is an increasing strain on the occluding plaque, FIG. 13. Uni-axial tensile experiments carried out to characterise arterial plaque have shown unique rupture strains exist for plaques of varying stiffness, FIG. 14. FIG. 13 illustrates the mechanics of the physical phenomena employed to define the geometry of the support scaffold. The highlighted region illustrates the region where these competing physical phenomena are at a minimum, thus maximising the effectiveness of the support scaffold. The stiffness of hard type plaque is employed in determining the inner support diameter as soft plaque type is capable of withstanding much greater strains, before rupture.

Hemodynamic instabilities during CAS have been ascribed to the consequences of direct carotid host tissue stimulation during balloon inflation [1], [2], [3], and can be described through the presence of hypotension, bradycardia and/or asystole. Mangin et al. (2003) found that CAS stimulates host tissue in all patients studied, resulting with a markedly decrease in heart rate and blood pressure. Gupta et al. (2005) reports that hemodynamic instability after CAS occurs in 29% to 51% of patients undergoing the procedure. Dangas et al. (2000) found that of 140 patients who underwent CAS, those with hypotension were more likely to suffer a minor stroke after the procedure [4]. Abou-Chebl et al. (2004) reports that from a total of 404, patients with haemodynamic instabilities had a significantly increased risk of stroke (odd ratio (OR)=2.6, 95% CI 1.2-5.9), myocardial infarction (OR=4.5, 95% CI 1.2-16.9) or death (OR=2.7, 95% CI 1.0-7.6) in the peri-operative period. The odds ratio for the combined endpoint of stroke, myocardial infarction or death was 3.6 (95% CI 1.8-6.9) in patients with haemodynamic instability. Reasons for hypotensive related stroke have been related to low flow and low blood pressure resulting from damage to the host tissue prior to CAS (during atherosclerotic build-up) or peri-procedural over-extension during CAS, (Gupta et al., 2005). The novelty of a two step dilation of occluded arterial vessel allows the reduction of overstretching of the host tissue, located in the carotid sinus. The first dilation step is instantaneous (similar to traditional current angioplasty balloon devices) but with small strains applied to the plaque preventing rupture, with a prolonged second dilation step, FIG. 15. For FIG. 15, the second dilation time step of the novel device is for example 4 magnitudes longer than that of traditional/current devices. This controlled second dilation step can be used with continuous monitoring of heart rate and blood pressure to minimise peri- and post-operative haemodynamic instabilities and other adverse events.

FIG. 16 demonstrates the velocity profiles along the centre of the artery as the blood passes from a full 5 mm vessel through the perfusion balloon and finally through a filter. Model A shows the perfusion balloon used in conjunction with a traditional catheter, model B shows the complete device with perfusion filter and model C shows the complete device upon deflation of the outer balloon before device retrieval. Each filters configuration was modelled as having high permeability, i.e. not blocked by emboli, and low permeability demonstrating the effect of a filter after capturing a sufficient amount of plaque. As filter A becomes clotted with emboli the velocity beyond the filter reduces dramatically. When deployed, the proposed device, model B, doesn't have this draw back because the filter enables the majority of blood flow through the device because it is emboli free. The clotting of the filter will affect the device once the outer balloon is deflated, model C, but as the filter becomes less permeable, the blood flow redirects itself through the perfusion balloon and an increase in blood velocity is seen, which is in stark contrast to model A.

FIG. 17 takes a closer look at the velocity contours and directions surrounding the filters that have been clotted with emboli for the 3 device configurations, highlighted in the box in FIG. 16. The restriction of blood flow is apparent in models A and C. However, model C permits the flow of emboli free blood through the device even if the filter becomes completely blocked. Therefore the device addresses both of the key design criteria associated with embolic devices, the filter can have a small pore size, so as to catch all required emboli, and it will enable continuous blood flow through the device to the brain. After traditional carotid angioplasty the embolic protection device remains within the vessel to capture post deflation plaque fragments that become disassociated from the wall. The issue surrounding this is the creation recirculating blood around the filter which increases blood residency times and can lead to thrombus formation.

The invention hence avoids the prior art problem of a catheter creating a blockage upon inflation. Not only can this prior approach result in an ischemic attack but it severely restricts the time that the physician has to propagate the artery. The invention enables antegrade blood flow during revascularisation, offering a potentially limitless timeframe within which to operate. The invention has the potential to enable protected deployment of further devices distal to the site of treatment due to the central passageway created by the inventions support system.

This support can in various embodiments be a shape-memory alloy, a mechanical support, an inner balloon or otherwise.

FIG. 18 illustrates a perspective cut-away view of an alternative embodiment of annular filter, 50, consisting of a filter mesh 51 folded over at 52 from a central passageway 53 and retained by shape memory alloy metal wires 54 connecting the catheter to the outer diameter of the annular filter. The shape memory alloy wires 54 are attached from a central catheter 55 to the outer diameter of the annular filter 51.

FIG. 19 illustrates a perspective cut-away view of a device 70 with a rib-cage balloon inner support structure 76, a coiled helical outer balloon 75, and a filter 71. The filter 71 has a capture region 72, a stem 73, and retainer wires 74.

FIG. 20 illustrates a two-balloon angioplasty device 90 having a filter 91 with an annular capture region 92, a central passageway stem 93, and support wires 94. There is a coiled outer balloon 95 and a coiled inner support balloon 96. This drawing illustrates how the pitch diameters can vary with respect to each other along the length of the balloons. This can be used as a mechanism to prevent the coils of each balloon slipping between one another.

FIG. 21 shows another balloon arrangement 110 for a device of the invention. There is an outer coiled balloon 111 and a coiled inner balloon 112. This embodiment demonstrates the different coil pitch angles that can be employed to provide additional support so that the balloons remain isolated and stable.

FIG. 22 illustrates a device 130 deployed in an artery. There is an outer coiled balloon 131, an inner coiled support balloon 132, filter support wires 133, and a filter capture region 134. The stem of the filter extends in-between the two balloons.

FIG. 23 illustrates a device 150 fully deployed in an artery. There is an outer coiled annular balloon 151, an inner coiled support balloon 152, and a filter 153 supported by an inflated ring 154.

Though there have been no randomised studies to date comparing CAS with and without EPD, the availability and employment of EPDs is important and has greatly reduced the risk of post-procedural complications during carotid angioplasty and stenting [5]. The device of the invention is a combination of an annular perfusion balloon and an annular perfusion filter. Prior art filters are designed to capture emboli fragments in the blood and also to permit blood to pass through the filter to distal arteries and the brain. Therefore the design of traditional filters involves a trade off in the functionality of both key design criteria through the design of the filter pore size and distribution. Larger pores increase filter permeabilty but could also permit emboli to pass through the filter which can be detrimental. However, even a filter that adheres to these criteria begins to fail as it collects emboli because the filters pores become blocked with captured emoboli which further restricts the flow of blood to the brain which can result in ischemic events. The invention provides a perfusion chamber through both the balloon and filter so that emboli-free blood can transverse, which is in contrast to traditional filters where the deposition of emboli coincides with a reduction in blood flow and an increase in the pressure drop in the vessel. The device of the invention prevents this from happening and can be designed with small pore size, to capture all potentionally detremental emboli, and have a constant supply of blood to the brain.

It will be appreciated that the invention avoids the major disadvantage with prior balloon catheters which is that in order to relieve a blockage in an artery it must first create one. When a typical prior device is inflated it blocks the whole artery and blood cannot flow during this time, particularly in patients with poor collateral blood flow.

In the invention the balloon inflates and expands the inner arterial wall with lesion, and the filter captures emboli released during the carotid angioplasty procedure. The filter extends fully between the perfusion balloon and the vessel wall, so that all dislodged emboli are captured. There is a channel that allows blood to flow to tissues distal to the blockage, maintaining antegrade flow. This may prevent the onset of an ischemic stroke or other perioperative neurological events. Due to there being a channel maintaining antegrade flow, the device would add a limitless time frame within which a surgeon can operate. This is advantageous in limiting the impact of the carotid angioplasty procedure on the host tissue and improving the health and state of the artery wall and the carotid sinus post procedure.

The device can be located at the site of the stenosis, encapsulating the plaque comprehensively, while also allowing antegrade blood flow at the site of the lesion. There is filter coverage of the entire plaque blockage during balloon pre-dilation, dilation and stent deployment so all factors which cause the plaque to be disturbed and generate emboli are protected by a safe and effective filter system.

In summary, the invention solves several problems of conventional treatment:

Blood Flow Restrictions:
Complete balloon occlusion of the vessel for extended periods of time lead to perioperative complications for the patient due to the reduced blood supply to cranial tissue
Filters get “clogged” with embolic fragments further preventing blood flow and increasing the pressure drop across the devices lessening blood supply to distal cranial tissues
Following the placement of a standard balloon and filter device, the pressure drop results in a transient episode of hypotension and further associated patient complications

### Artery Wall Implications:

Controlled inflation of the device reduces the strain rate incurred by the atherosclerotic lesion, reducing the potential of emboli.
Gradual strain of the arterial wall reduces the likelihood of elastic recoil.
Gradual stretch of the carotid sinus minimizes the likelihood of haemodynamics instabilities such as post-surgery hypotension.

The invention is not limited to the embodiments described but may be varied in construction and detail. For example, inflation may be with gas or a liquid medium, depending on the application. Also, the invention can be employed as either a stent delivery system whereby a stent can be placed on the outer balloon or a self expanding stent can be deployed after the balloon is expanded, or an endograft delivery system whereby the balloon ensures endograft expansion, placement and fixation. In various embodiments the filter support wires or filter support balloon and filter net are bonded or welded to the inner surface of the outer balloon.

### REFERENCES

[1] Abou-Chebl A., Gupta R., Bajzer C. T., (2004). Consequences of hemodynamic instability after carotid artery stenting. J Am Coll Cardiol. 43, 1:A20-A21
[2] Mangin L.; Medigue C.; Merle J-C.; Macquin-Mavier I.; Duvaldestin P.; Monti A.; Becquemin J-P. (2003) Cardiac autonomic control during balloon carotid angioplasty and stenting. Canadian Journal of Physiology and Pharmacology, Volume 81, Number 10, pp. 944-951
[3] Gupta, R., Horowitz, M., Jovin, T. G., (2005). Haemodynamic instability after carotid artery angioplasty and stent placement: a review of the literature. Neurosurg Focus 18 (1): E6
[4] Dangas G., Laird J. R., Satler L. F., Mehran R., Mintz G. S., Larrain G., Lansky A. J., Gruberg L., Parsons E M, Laureno R (2000). Postprocedural hypotension after carotid artery stent placement: predictors and short- and long-term clinical outcomes. Radiology, 215: 677-683
[5] Kastrup, A., Nägele, T., Gröschel, K., Schmidt, F., Vogler, E., Schulz, J., Ernemann, U., (2006). Incidence of New Brain Lesions after Carotid Stenting with and without Cerebral Protection. Stroke, 37: 2312-2316.

1. A vascular device comprising:
a perfusion balloon having, when inflated, a through-hole for blood flow, and a distal filter for trapping emboli,
wherein the filter comprises a filtering portion which is configured to filter an annular cross-sectional area extending from a vessel wall while leaving an un-filtered central passageway.

2. The device as claimed in claim 1, wherein the filtering portion is configured to, when deployed, extend distally from the perfusion balloon and then in a direction which is at least partly radial, and then in a direction which is at least partly proximal.

3. The device as claimed in claim 1, wherein the filtering portion is substantially frusto-conical in shape when deployed.

4. The filter device as claimed in claim 1, wherein the filter has a proximal stem which is attached to the perfusion balloon or a support for the perfusion balloon.

5. The device as claimed in claim 1, wherein the filtering portion has an asymmetrical edge for improved retrieval to enable improved device deliverability and extraction.

6. The device as claimed in claim 1, wherein the filter comprises a retainer adapted to retain an end of the filtering portion in an in-use position abutting a vessel wall.

7. The device as claimed in claim 6, wherein the retainer comprises members extending from the perfusion balloon or a support for the perfusion balloon.

8. The device as claimed in claim 6, wherein the retainer comprises members extending from the perfusion balloon or a support for the perfusion balloon; and wherein the members include wires.

9. The device as claimed in claim 6, wherein the retainer is adapted to apply a radially outward bias to urge the filter to abut a vessel wall.

10. The device as claimed in claim 6, wherein the retainer is adapted to apply a radially outward bias to urge the filter to abut a vessel wall; and wherein the retainer comprises cantilevered members.

11. The device as claimed in claim 6, wherein the retainer is ring-shaped having a configuration conforming to a vessel perimeter.

12. The device as claimed in claim 6, wherein the retainer is ring-shaped having a configuration conforming to a vessel perimeter; and wherein the retainer is inflatable.

13. The device as claimed in claim 6, wherein the retainer is ring-shaped having a configuration conforming to a vessel perimeter; and wherein the retainer is inflatable; and wherein the retainer is linked with the balloon for simultaneous inflation.

14. The device as claimed in claim 1, further comprising an internal support for the perfusion balloon.

15. The device as claimed in claim 14, wherein the support is adapted to allow gradual and controlled inflation of the perfusion balloon.

16. The device as claimed in claim 14, wherein the support comprises a scaffold structure.

17. The device as claimed in claim 14, wherein the support comprises a scaffold structure; and wherein the scaffold structure comprises elements of a shape memory material.

18. The device as claimed in claim 14, wherein the support comprises at least one element in the form of opposed arches.

19. The device as claimed in claim 14, wherein the support comprises an element in the form of a coil.

20. The device as claimed in claim 14, wherein the support comprises an inner balloon adapted to fit within the perfusion balloon.

21. The device as claimed in claim 14, wherein the support comprises an inner balloon adapted to fit within the perfusion balloon; and wherein the inner and perfusion balloons are coiled, the coil pitches being in or out of phase.

22. The device as claimed in claim 1, wherein the device is adapted to support a stent around the perfusion balloon and a mechanism for delivery of the stent by inflation of the perfusion balloon.

23. A method of performing a procedure using a vascular device comprising a perfusion balloon having, when inflated, a through-hole for blood flow, and a distal filter for trapping emboli, the filter being configured to filter an annular cross-sectional area extending from a vessel wall while leaving an un-filtered central passageway, the method comprising the steps of:
inserting the device into a vessel,
inflating the perfusion balloon while allowing blood to flow through the through-hole,
the filter capturing emboli which are dislodged from the vessel wall while allowing un-impeded blood flow through the central passageway

24. The method as claimed in claim 23, wherein the filter is supported by retainers to engage the vessel wall.

25. The method as claimed in claim 24, wherein the retainer is inflatable and is inflated together with the perfusion balloon so that it engages around the vessel wall.
